(19) United States
(12) Patent Application Publication
(10) Pub. No.: US 2016/0193372 A1
(43) Pub. Date: Jul. 7, 2016

(54) METHODS OF IDENTIFYING SURROGATE IMAGING AGENTS FOR ALPHA EMITTER RADIOPHARMACEUTICALS AND THEIR USE IN TREATMENT OF DISEASE

(71) Applicant: THE JOHNS HOPKINS UNIVERSITY, Baltimore, MD (US)

(72) Inventors: George Sgouros, Ellicott City, MD (US); Robert Hobbs, Baltimore, MD (US); Hong Song, Baltimore, MD (US)

(21) Appl. No.: 14/647,890

(22) PCT Filed: Nov. 27, 2013
(86) PCT No.: PCT/US2013/072147

§ 371 Date: May 28, 2015

Related U.S. Application Data
(60) Provisional application No. 61/731,170, filed on Nov. 29, 2012.

(51) Int. Cl. A61K 51/10

(52) U.S. Cl. CPC A61K 51/1096

(57) ABSTRACT

In accordance with one or more embodiments, the present invention provides a series of identified radiopharmaceuticals herein termed “imaging surrogates” whose pharmacokinetic behavior matches that of the alphaRPT for several alphaRPTs currently under investigation as cancer therapeutics. Methods for identifying imaging surrogates are also provided. These imaging surrogates can be used to implement a treatment planning approach to radiopharmaceutical therapy of alpha-emitters, enabling treatment to the maximum tolerated organ absorbed dose for individual patients.

### REFERENCE TO RELATED APPLICATIONS

This application claims the benefit of U.S. Provisional Patent Application No. 61/731,170, filed on Nov. 29, 2012, which is hereby incorporated by reference for all purposes as if fully set forth herein.

### STATEMENT OF GOVERNMENTAL INTEREST

This invention was made with government support under grant nos. CA116477 and CA113797 awarded by the National Institutes of Health. The government has certain rights in the invention.

### BACKGROUND OF THE INVENTION

Medical radionuclide imaging (e.g., Nuclear Medicine) is a key component of modern medical practice. This methodology involves the administration, typically by injection, of tracer amounts of a radioactive substance (e.g., radiotracer agents, radiotherapeutic agents, and radiopharmaceutical agents), which subsequently localize in the body in a manner dependent on the physiologic function of the organ or tissue system being studied. The radiotracer emissions, most commonly gamma photons, are imaged with a detector outside the body, creating a map of the radiotracer distribution within the body. When interpreted by an appropriately trained physician, these images provide information of great value in the clinical diagnosis and treatment of disease. A longitudinal set of such images may also be used to perform dosimetry calculations using methods that have been previously described.

Recent advances in diagnostic imaging, such as magnetic resonance imaging (MRI), computerized tomography (CT), single photon emission computerized tomography (SPECT), and positron emission tomography (PET) have made a significant impact in cardiology, neurology, oncology, and radiology. Although these diagnostic methods employ different techniques and yield different types of anatomic and functional information, this information is often complementary in the diagnostic process. The combination of anatomical data (e.g., from MRI and/or CT) and pharamacokinetic data (e.g., from SPECT and/or PET) has also permitted patient-specific 3-D, imaging based dosimetry calculations.

Radionuclides are generally classified as either being diagnostic or therapeutic in their application. Although diagnostic imaging agents have historically been a mainstay in the nuclear pharmacy industry, during the past decade there has been increased interest in the development and use of therapeutic radiotherapeutic agents. This shift in focus has been elicited primarily from research involving combining radionuclides with sophisticated molecular carriers. Because of radiation's damaging effect on tissues, it is important to target the biodistribution of radiopharmaceuticals as accurately as possible. Generally speaking, PET uses imaging agents labeled with the positron-emitters such as ¹⁸F, ¹¹C, ¹³N and ¹⁵O, ⁷⁵Br, ⁷⁶Br, ¹²⁴I, ⁶⁸Ga, ⁶⁴Cu and ⁹⁸Zr, SPECT uses imaging agents labeled with the single-photon-emitters such as ²⁰¹Tl, ^{99m}Tc, ¹²³I, ¹¹¹In and ¹³¹I.

Alpha-particle emitter radiopharmaceutical therapy (RPT) uses alpha-emitting radionuclides to kill the targeted cell or population of cells, such as cancer or tumor cells. Examples of such alpha emitters include, for example, ²¹³Bi labeled agents and ²²⁵Ac labeled agents or collectively, “alphaRPTs,” and they are usually delivered at activity levels that are too low for quantitative SPECT imaging, alternatively the emission properties of promising alpha-particle emitters may be inappropriate for imaging. The inability to collect quantitative SPECT imaging is a key potential obstacle to a dosimetry-driven, patient-specific implementation of alpha-emitter RPT. Even when quantitative SPECT imaging is possible, the resolution currently achievable in human imaging does not provide the activity resolution required to obtain absorbed dose distributions that account for the short range of alpha-particle emissions (i.e., at the microscopic level), which would be useful in developing better treatment regimens for cancer and other diseases.

For example, osteosarcoma (OS) is the most common primary bone malignancy in children and adolescents. The 10-year survival in patients with metastatic OS is 25%; following recurrence, it is 20%. Treatment of high risk (i.e., metastatic at diagnosis or recurrent) OS has remained essentially unchanged over the past 20 years. High intensity chemotherapy has not improved the prognosis of metastatic or recurrent OS. The advances in biologic and pathway inhibition therapy that have been observed for other cancers have not been seen for OS. RPT using ¹⁵³Sm-EDTMP has also been investigated in the treatment of OS. This agent targets mineralizing or proliferating bone matrix. At very high administered activities, it has yielded pain palliation but minimal therapeutic efficacy. The well-established radioresistance of OS partially explains these observations. Another explanation is that ¹⁵³Sm-EDTMP and other bone seeking agents are fundamentally limited since they do not target soft tissue lesions.

Therefore, there still exists an unmet need to develop surrogate RPT imaging agents for whose pharmacokinetic profile is significantly similar to that of the alphaRPT of interest for cancer therapeutics, such as OS, and their use in other diseases.

### SUMMARY OF THE INVENTION

In accordance with one or more embodiments, the present invention provides methods for identifying radiopharmaceuticals herein termed “imaging surrogates” whose pharmacokinetic behavior matches that of the alphaRPT for several alphaRPTs currently under investigation as cancer therapeutics. These identified imaging surrogates can then be used to implement a treatment planning approach to radiopharmaceutical therapy of alpha-emitters, enabling treatment to the maximum tolerated organ absorbed dose for individual patients.

In accordance with one or more embodiments, the present invention provides an imaging surrogate composition comprising at least detectable moiety conjugated to a targeting moiety, wherein the imaging surrogate composition has a pharmacokinetic profile which is significantly similar to an alpha-emitting radionuclide conjugated to the same targeting moiety (alphaRPT).

In accordance with an embodiment, the present invention provides for the use of the imaging surrogate compositions described herein to determine the appropriate dosage level of an alphaRPT of interest in a subject comprising: a) administering the imaging surrogate to the subject; b) measuring the pharmacokinetic profile of the imaging surrogate in the subject using imaging; and c) if necessary, performing pharmacokinetic modeling to account for differences between the imaging surrogate and the alphaRPT; and d) using the information from b) and/or c) to determine the appropriate dosage level of the alphaRPT of interest for the subject.

In accordance with another embodiment, the present invention provides for the use of the imaging surrogate compositions described herein in the treatment of a disease in a subject comprising: a) determining an appropriate dosage level of an alphaRPT of interest in a subject comprising administering an imaging surrogate to the subject; b) measuring the pharmacokinetic profile of the imaging surrogate in the subject using imaging; c) if necessary, performing pharmacokinetic modeling to account for differences between the imaging surrogate and the alphaRPT; d) determining the appropriate dosage level of the alphaRPT of interest for the subject; and e) administering to the subject the alphaRPT of interest at the dosage level determined in d) to treat the disease in the subject.

In accordance with an embodiment, the present invention provides a method for determining an appropriate dosage level of an alphaRPT of interest in a subject comprising: a) administering an imaging surrogate composition to the subject, wherein the imaging surrogate comprises at least one detectable moiety conjugated to a targeting moiety, and wherein the imaging surrogate composition has a pharmacokinetic profile which is significantly similar to an alphaRPT comprising the same targeting moiety labeled with alpha-emitting isotope; b) measuring the pharmacokinetic profile of the imaging surrogate composition in the subject using imaging; c) if necessary, performing pharmacokinetic modeling to account for differences between the imaging surrogate composition and the alphaRPT; and d) determining the appropriate dosage level of the alphaRPT of interest for the subject.

In accordance with another embodiment the present invention provides a method for determining an appropriate dosage level of an alphaRPT of interest in a subject comprising: a) administering an imaging surrogate composition to the subject, wherein the imaging surrogate comprises ¹¹¹In or ⁶⁸Ga or ⁸⁹Zr conjugated to a targeting moiety, and wherein the imaging surrogate composition has a pharmacokinetic profile which is significantly similar to an alphaRPT conjugated to the same targeting moiety labeled with an alpha-emitting isotope selected from the group consisting of ²¹³Bi, ²²⁵Ac and ²¹²Pb; b) measuring the pharmacokinetic profile of the imaging surrogate in the subject using imaging; c) if necessary, performing pharmacokinetic modeling to account for differences between the imaging surrogate and the alphaRPT; and d) determining the appropriate dosage level of the alphaRPT of interest for the subject.

In accordance with another embodiment the present invention provides a method for determining an appropriate dosage level of an alphaRPT of interest in a subject comprising: a) administering an imaging surrogate composition to the subject, wherein the imaging surrogate comprises ¹³¹I conjugated to a targeting moiety, and wherein the imaging surrogate composition has a pharmacokinetic profile which is significantly similar to an alphaRPT conjugated to the same targeting moiety labeled with ²¹¹At; b) measuring the pharmacokinetic profile of the imaging surrogate in the subject using imaging; c) if necessary, performing pharmacokinetic modeling to account for differences between the imaging surrogate and the alphaRPT; and d) determining the appropriate dosage level of the alphaRPT of interest for the subject.

In accordance with another embodiment the present invention provides a method for determining an appropriate dosage level of an alphaRPT of interest in a subject comprising: a) administering an imaging surrogate composition to the subject, wherein the imaging surrogate comprises ¹³¹I conjugated to a targeting moiety, and wherein the imaging surrogate composition has a pharmacokinetic profile which is significantly similar to an alphaRPT conjugated to the same targeting moiety labeled with ²¹¹At; b) measuring the pharmacokinetic profile of the imaging surrogate in the subject using imaging; c) if necessary, performing pharmacokinetic modeling to account for differences between the imaging surrogate and the alphaRPT; and d) determining the appropriate dosage level of the alphaRPT of interest for the subject.

In accordance with another embodiment the present invention provides a method for determining an appropriate dosage level of an alphaRPT of interest in a subject comprising: a) administering an imaging surrogate composition to the subject, wherein the imaging surrogate comprises ⁸⁹Zr conjugated to a targeting moiety, and wherein the imaging surrogate composition has a pharmacokinetic profile which is significantly similar to an alphaRPT conjugated to the same targeting moiety labeled with ²²⁵Ac; b) measuring the pharmacokinetic profile of the imaging surrogate in the subject using imaging; c) if necessary, performing pharmacokinetic modeling to account for differences between the imaging surrogate and the alphaRPT; and d) determining the appropriate dosage level of the alphaRPT of interest for the subject.

In accordance with a further embodiment, the present invention provides a method of treatment of a disease in a subject comprising: a) administering an imaging surrogate composition to the subject, wherein the imaging surrogate comprises at least one detectable moiety conjugated to a targeting moiety, and wherein the imaging surrogate composition has a pharmacokinetic profile which is significantly similar to an alphaRPT comprising the same targeting moiety labeled with alpha-emitting isotope; b) measuring the pharmacokinetic profile of the imaging surrogate in the subject using imaging; c) if necessary, performing pharmacokinetic modeling to account for differences between the imaging surrogate and the alphaRPT; d) using the information from b) and/or c) to determine the appropriate dosage level of the alphaRPT of interest for the subject; and e) administering to the subject the alphaRPT of interest at the dosage level determined in d) to treat the disease in the subject.

### BRIEF DESCRIPTION OF THE DRAWINGS

FIG. 1 depicts time-activity curves for blood and normal organs of ²¹³Bi- and ¹¹¹In-7.16.4 in tumor-free mice (4 mice/time-point/radionuclide; 2 hour time-point not included for ¹¹¹In); error bars are the standard deviation. ²¹³Bi curves are not decay corrected since physical decay must be included during integration of these curves for dosimetry. To compare with ²¹³Bi, the ¹¹¹In results have been adjusted to reflect physical loss consistent w/the 45.6 min half-life of ²¹³Bi.

FIG. 2 depicts time-activity data for blood and normal organs of ²²⁵Ac- and ¹¹¹In-radiolabeled peptide in one representative tumor-free mice healthy mouse per time point.

FIG. 3 is a flow chart depicting an algorithm of the present invention used to assess the imaging surrogate dosimetry of the present invention as a surrogate for the alphaRPT of interest.

FIG. 4A is a schematic depiction of RPT dosing by a previously defined fixed administered activity (AA) for the dose limiting toxicity (AADLT) may be appropriate for patient 1 but substantially under doses patient 2 who exhibits more rapid clearance kinetics. In this case the activity corresponding to dose-limiting organ (DLO) toxicity is higher. AA-based dosing can under (or over) dose by not accounting for patient differences in pharmacokinetics (e.g, λ1 vs λ2).

FIG. 4B is a schematic depiction of the absorbed dose (AD)-based RPT methods of the present invention. In AD-based dosing, patient-specific dosimetry provides AA to deliver ADDLT. This accounts for pharmacokinetic and other differences among patients.

FIG. 5 is taken from (Semin. Oncol. 2003; 30(2):31-38) and illustrates the variability in AA across a large patient population when treatment is customized to deliver the dose-limiting absorbed dose to the DLO. In the example, shown, the most frequently applicable AA value (˜100 mCi) would under or overtreat a substantial fraction of the treated patients. This would be unacceptable for pediatric cancer patients.

FIG. 6 is a schematic diagram of ⁸⁹Zr-DFO-trastuzumab preparation.

FIG. 7 is a schematic diagram of ²²⁵Ac-DOTA-trastuzumab preparation.

FIG. 8 is an illustration of the phase I/II type protocol used with the inventive methods.

### DETAILED DESCRIPTION OF THE INVENTION

In accordance with one or more embodiments, the present invention provides an imaging surrogate composition comprising at least one at least one detectable moiety conjugated to a targeting moiety, wherein the imaging surrogate composition has a pharmacokinetic profile which is significantly similar to an alpha-emitting radionuclide conjugated to the same targeting moiety (alphaRPT).

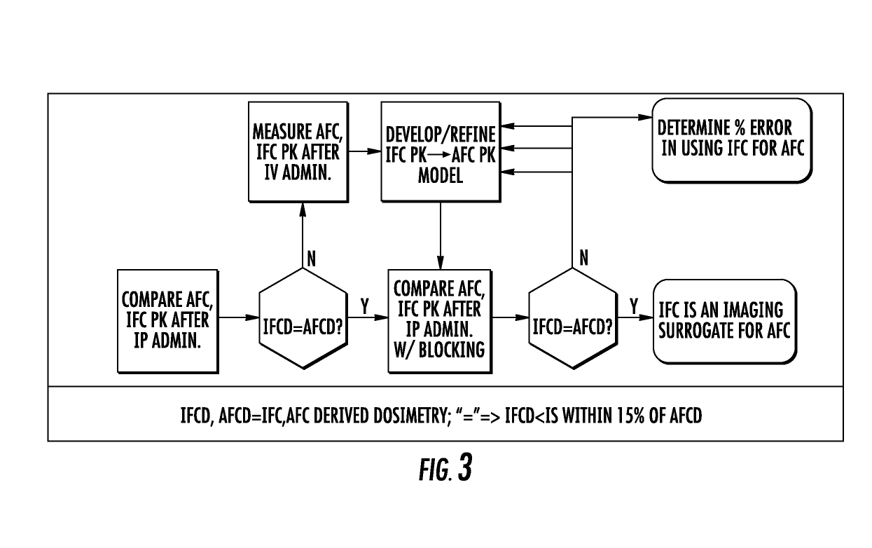

In accordance with one or more embodiments of the compositions and methods of the present invention, absorbed doses are scaled by the activity level required for organ toxicity or anti-tumor efficacy. AlphaRPT using peptides (or other targeting ligands) can be envisioned in which an imaging peptide conjugate (iPC) is paired with an alpha-emitting peptide conjugate (aPC). The paired iPC and aPC measurements are compared to assess the fidelity of iPC as a surrogate for aPC without pharmacokinetic (PK) modeling by insuring that iPC derived aPC dosimetry (iPCD) is within a range from about 1 to 15% of directly calculated aPC dosimetry (aPCD) to the dose-limiting organ. Should PK modeling be required to use IPC as a surrogate for aPC, additional combined iPC+aPC PK will be obtained following IV injection of iPC+aPC in preclinical models. These additional PK data will be used to develop the iPC to aPC PK model. Model validation is accomplished by using the model to predict aPC PK given iPC PK as input. The model is developed using aPC injection data then tested to see if iPC input predicts aPC dosimetry. The iPC credentialing approach used in accordance with the methods of the present invention is summarized in FIG. 3.

It will be understood by those of ordinary skill in the art that the terms “imaging peptide conjugate (iPC)” and “alpha-emitting peptide conjugate (aPC)” are used for descriptive purposes, and are not limited to compositions comprising peptide conjugates. Other targeting ligands with specificity for a target tissue or organ can be conjugated to the imaging conjugate or alpha-emitting conjugate molecule. These ligands can include small molecules, such as drugs for example, which bind a specific receptor.

As used herein, the term “alpha-emitting peptide conjugate” is synonymous with the term “alphaRPT” and can be used interchangeably.

By “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. Specific radioactive labels include most common commercially available isotopes including, for example, ³H, ¹¹C, ¹³C, ¹⁵N, ¹⁸F, ¹⁹F, ¹²³I, ¹²⁴I, ¹²⁵I, ¹³¹I, ⁸⁶Y, ⁸⁹Zr, ¹¹¹In, ^{94m}Tc, ^{99m}Tc, ⁶⁴Cu and ⁶⁸Ga, including those which can emit at least one or more photons. Suitable dyes include any commercially available dyes such as, for example, 5(6)-carboxyfluorescein, IRDye 680RD maleimide or IRDye 800CW, ruthenium polypyridyl dyes, and the like.

As used herein, the term “targeting moiety” is defined as any polypeptide, protein, nucleic acid based construct, nanovesicle, nanoparticle, whether synthetic or naturally derived, or fragment thereof which selectively binds to a particular cell type in a host through recognition of a cell-type specific (e.g. tumor cell or other host cell or population of cells) marker (e.g., antigen or receptor. In an embodiment, the cell targeting moiety can be an antibody or peptide. Other proteins, enzymes and growth factors can also be targeting moieties. Targeting moieties can also be defined as an antibody or fragment thereof which selectively binds to a particular cell type in a host through recognition of a cell surface antigen. Preferred cell targeting antibodies are specific for solid tumors and cancer cells.

In accordance with an embodiment, the present invention provides a method of treating or preventing cancer in a subject using the methods of alphaRPT described herein.

In accordance with another embodiment, the cancer being treated can be any cancer where the disease presents as one or more solid tumors. Examples of such cancers include, alveolar rhabdomyosarcoma, bone cancer, brain cancer, breast cancer, cancer of the anus, anal canal, or anorectum, cancer of the eye, cancer of the intrahepatic bile duct, cancer of the neck, gallbladder, or pleura, cancer of the nose, nasal cavity, or middle ear, cancer of the oral cavity, cancer of the vulva, colon cancer, esophageal cancer, cervical cancer, gastrointestinal carcinoid tumor, hypopharynx cancer, kidney cancer, larynx cancer, hepatocellular carcinoma, lung cancer, malignant mesothelioma, melanoma, multiple myeloma, nasopharynx cancer, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In a preferred embodiment, the cancer is osteosarcoma (OS).

In an embodiment, the methods of the present invention can be used for developing alphaRPT treatments for OS. A treatment-planning based approach to alphaRPT is essential to maximizing efficacy without inducing prohibitive toxicity. This is especially important since OS patients are typically between 10 to 25 years old. The large range in height, weight and potential tumor burden requires that alphaRPT be administered based on the absorbed dose (AD) to the dose-limiting organ (DLO) rather than on administered activity (AA) or AA per weight or surface area. AA-based treatment will deliver either a sub-optimal tumor AD or a morbidity-inducing AD to the DLO because there will be age and tumor-burden related differences in tumor targeting and clearance of alphaRPT. By adopting a treatment planning approach using the methods of the present invention, this is avoided.

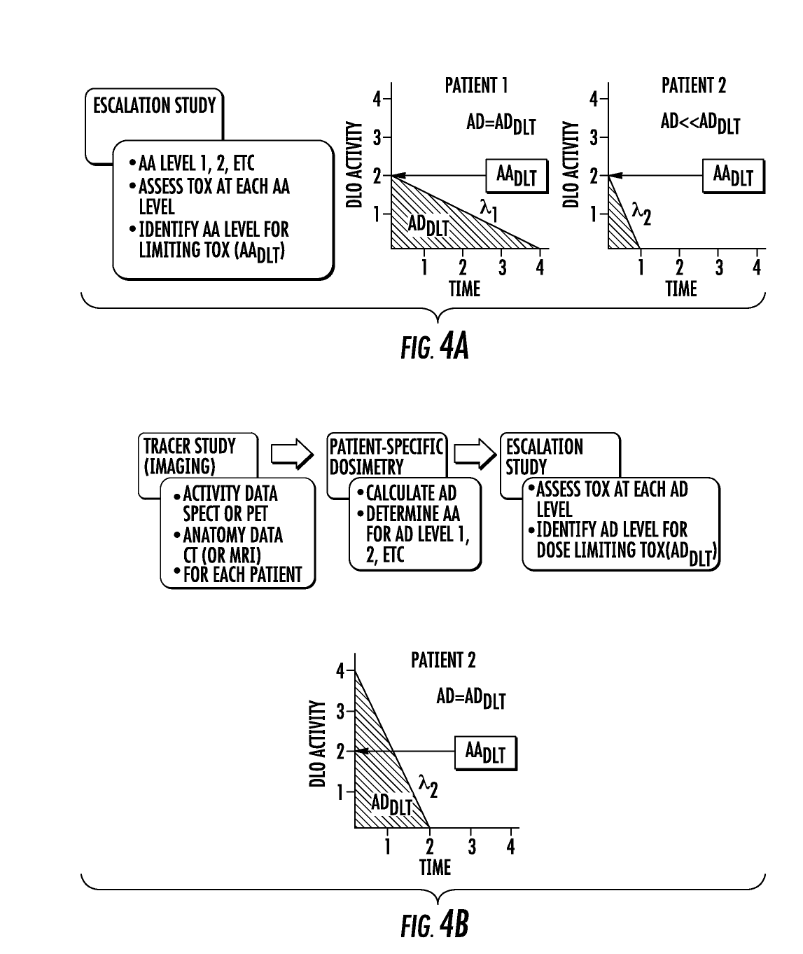

The imaging and PK data collection derived from the methods of the present invention is integral to adopting a patient-specific, treatment-planning approach to implementing alphaRPT in human patients with high risk OS. The significance of this may be understood by comparison with chemotherapy dosing. Chemotherapy is administered on a per body weight or body surface area basis. Typically the dose used is obtained from a phase I dose escalation trial used to define the MTD. The MTD is typically defined by the response of a limited number of patients, typically 6 patients in a dose group. This MTD is then used to treat all other patients in Phase II and subsequent trials. This approach does not account for differences in drug clearance, metabolism or PK in different patients. The outcome of such an approach is that patients will either be underdosed (if a dose is chosen to avoid toxicity across a majority of patients) or they will be overdosed and experience toxicity. In alphaRPT it is possible to collect PK and imaging data to calculate tumor and DLO absorbed dose from a given AA. Thus AA can be adjusted to customize treatment for each patient to deliver the maximum possible absorbed dose to tumor without exceeding the absorbed dose to DLO that leads to toxicity (FIGS. 4A-B).

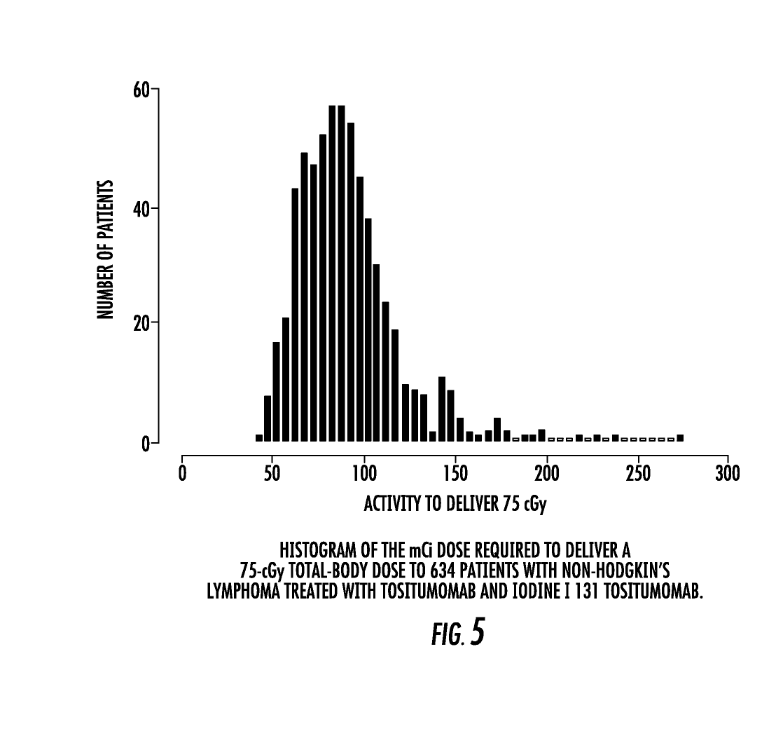

OS patients will range in age, weight, height, and tumor burden. Because of this potential variability, alphaRPT treatment planning using the methods of the present invention is essential to assure that treatment is appropriate for each individual patient. This concept is well recognized and accepted in external beam radiotherapy. It has been implemented for the treatment of Non-Hodgkin's Lymphoma using ¹³¹I-conjugated Ab and whole-body absorbed dose as a surrogate for the absorbed dose to marrow, the dose-limiting organ. The impact of this approach is illustrated in FIG. 5.

It will be understood by those of ordinary skill that therapy with α-emitters is fundamentally different from other cancer treatments. No other modality can deliver high energy, short-range particles to targeted cells. In contrast to biologic (or pathway inhibition) therapy wherein the fundamental cancer biology, or for that matter, any disease biology, must be understood in order to find therapeutic agents, whereas alphaRPT depends upon identifying a tumor associated cell-surface target. Cell kill depends far more on adequate delivery of α-particles and on the physics of high linear energy transfer radiation than on the biology of the tumor cell. More importantly, alphaRPT has not been previously investigated for the treatment of OS. Examples of alpha-emitting radionuclides useful in the compositions and methods of the present invention include ¹⁴⁹Tb, ²¹¹At, ²¹²Bi, ²¹³Bi, ²¹²Pb, ²²³Ra, ²²⁴Ra, ²²⁵Ac, ²²⁶Ra, and ²²⁷Th.

In an embodiment, the term “administering” means that the compounds of the present invention are introduced into a subject, preferably a subject receiving treatment for a disease, and the compounds are allowed to come in contact with the one or more disease related cells or population of cells in vivo. In some embodiments the host cell or population of cells in the host can be any cell or population of cells that can be selectively bound by the targeting moiety described above. One of ordinary skill in the art would understand the host cells can be cancer cells. In other embodiments, the host cell or population of cells could be immunological cells, such as B cells, T cells, including tumor infiltrating lymphocytes, or, for example, other cells for which alphaRPT is appropriate.

In accordance with a further embodiment, the present invention provides a method of treatment of a disease in a subject comprising: a) administering an imaging surrogate composition to the subject, wherein the imaging surrogate comprises at least one detectable moiety conjugated to a targeting moiety, and wherein the imaging surrogate composition has a pharmacokinetic profile which is significantly similar to an alphaRPT comprising the same targeting moiety labeled with alpha-emitting isotope; b) measuring the pharmacokinetic profile of the imaging surrogate in the subject using imaging; c) if necessary, performing pharmacokinetic modeling to account for differences between the imaging surrogate and the alphaRPT; d) using the information from b) and/or c) to determine the appropriate dosage level of the alphaRPT of interest for the subject; and e) administering to the subject the alphaRPT of interest at the dosage level determined in d) to treat the disease in the subject.

Non-limiting examples of non-cancer diseases in which host cells can be targeted using the methods of the present invention include, autoimmune diseases, trigeminal neuralgia, acoustic neuromas, severe thyroid eye disease, pterygium, pigmented villonodular synovitis, atherosclerotic plaque, and prevention of keloid scar growth, vascular restenosis, and heterotopic ossification.

In an embodiment, the detectable moiety of the imaging surrogate can be any radioisotope used in the art which can be detected in vivo or using imaging analysis which is labeled to targeting moiety whose pharmacokinetics are similar to those of the alphaRPT of interest. Examples of such radioisotopes include but are not limited to, ²⁰¹Tl, ^{99m}Tc, ¹²³I, ¹¹¹In, ⁶⁸Ga, ¹²⁴I, ⁸⁹Zr and ¹³¹I.

In some other embodiments, the detectable moiety of the imaging surrogate can be a fluorescent dye. The dyes may be emitters in the visible or near-infrared (NIR) spectrum. Known dyes useful in the present invention include carbocyanine, indocarbocyanine, oxacarbocyanine, thüicarbocyanine and merocyanine, polymethine, coumarine, rhodamine, xanthene, fluorescein, borondipyrromethane (BODIPY), Cy5, Cy5.5, Cy7, VivoTag-680, VivoTag-S680, VivoTag-S750, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

Organic dyes which are active in the NIR region are known in biomedical applications. However, there are only a few NIR dyes that are readily available due to the limitations of conventional dyes, such as poor hydrophilicity and photostability, low quantum yield, insufficient stability and low detection sensitivity in biological system, etc. Significant progress has been made on the recent development of NIR dyes (including cyanine dyes, squaraine, phthalocyanines, porphyrin derivatives and BODIPY (borondipyrromethane) analogues) with much improved chemical and photostability, high fluorescence intensity and long fluorescent life. Examples of NIR dyes include cyanine dyes (also called as polymethine cyanine dyes) are small organic molecules with two aromatic nitrogen-containing heterocycles linked by a polymethine bridge and include Cy5, Cy5.5, Cy7 and their derivatives. Squaraines (often called Squarylium dyes) consist of an oxocyclobutenolate core with aromatic or heterocyclic components at both ends of the molecules, an example is KSQ-4-H. Phthalocyanines, are two-dimensional 18π-electron aromatic porphyrin derivatives, consisting of four bridged pyrrole subunits linked together through nitrogen atoms. BODIPY (borondipyrromethane) dyes have a general structure of 4,4′-difluoro-4-bora-3a, 4a-diaza-s-indacene) and sharp fluorescence with high quantum yield and excellent thermal and photochemical stability.

As used herein, the term “alphaRPT of interest” means any targeting moiety labeled or conjugated to an alpha-emitting radioisotope.

In some embodiments, the imaging surrogate compositions described herein can include imaging surrogates wherein the at least one or more positron emitting radionuclides or at least one or more photon-emitting radionuclides of the imaging surrogate composition is selected from the group consisting of ¹⁸F, ¹¹C, ¹³N and ¹⁵O, ⁷⁵Br, ⁷⁶Br, ¹²⁴I, ²⁰¹Tl, ^{99m}Tc, ¹²³I, ¹¹¹In, ⁶⁸Ga, ⁸⁹Zr and ¹³¹I.

In accordance with an embodiment the imaging surrogate composition of the present invention comprises ¹¹¹In and has a pharmacokinetic profile which is significantly similar to an alphaRPT comprising ²¹³Bi, or ²²⁵Ac, or ²¹²Pb.

In accordance with another embodiment, the imaging surrogate composition of the present invention is selected from the group consisting of ¹³¹I, ¹²³I, or ¹²⁴I, and has a pharmacokinetic profile which is significantly similar to an alphaRPT comprising ²¹¹At.

In accordance with a further embodiment, the imaging surrogate composition of the present invention comprises ¹³¹I and has a pharmacokinetic profile which is significantly similar to an alphaRPT comprising ²¹¹At.

In accordance with another embodiment, the imaging surrogate composition of the present invention comprises ⁸⁹Zr and has a pharmacokinetic profile which is significantly similar to an alphaRPT comprising ²²⁵Ac.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of a disease in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof.

As used herein the term “significantly similar” means that the pharmacokinetic profile of the imaging surrogate is within about 1, 2, 3, 5, 10, 15 or 20% of the pharmacokinetic profile of the alphaRPT of interest. The % value is not limiting, depending upon the sensitivity of the dose-limiting organ and the dose-response relationship values that are greater or lesser could also qualify as “significantly similar.” In a preferred embodiment, the pharmacokinetic profile of the imaging surrogate is within about 10-15% of the pharmacokinetic profile of the alphaRPT of interest.

As used herein, the term “detection” “imaging” or “radiodetection” in some embodiments, means the use of certain properties of isotopes and the energetic particles emitted from radioactive material to obtain pharmacokinetic information. In addition, the term “scintigraphy” means a diagnostic test in which a two-dimensional image of a body having a radiation source is obtained through the use of radioisotopes. A radioactive chemical is injected into the artery or vein of a patient which then concentrates in the target cells or organ of interest. By placing a camera that senses radioactivity over the body, an image of the target cells or organ of interest can be created. The particles can be detected by suitable devices such as gamma cameras, positron emission tomography (PET) machines, single photon emission computed tomography (SPECT) machines, planar imaging devices and the like.

In some other embodiments, the term “detection” or “imaging” means the detection of photons from fluorescent or NIRF dyes using known detection methods, including, for example, laser fluorescent confocal microscope imaging.

In an embodiment, the compositions can include alphaRPT and imaging surrogates in conjunction with a carrier. The carrier is preferably a pharmaceutically acceptable carrier. With respect to pharmaceutical compositions, the carrier can be any of those conventionally used and is limited only by chemico-physical considerations, such as solubility and lack of reactivity with the active compound(s), and by the route of administration. The pharmaceutically acceptable carriers described herein, for example, vehicles, adjuvants, excipients, and diluents, are well-known to those skilled in the art and are readily available to the public.

The choice of carrier will be determined in part by the particular alphaRPT and imaging surrogates used as well as by the particular method used to administer the alphaRPT and imaging surrogates. Accordingly, there are a variety of suitable formulations of the pharmaceutical composition of the invention. The following formulations for parenteral, subcutaneous, intravenous, intramuscular, and intraarterial, administration are exemplary and are in no way limiting. More than one route can be used to administer the alphaRPT and imaging surrogates, and in certain instances, a particular route can provide a more immediate and more effective response than another route.

Preservatives and buffers may be used. In order to minimize or eliminate irritation at the site of injection, such compositions may contain one or more nonionic surfactants having a hydrophile-lipophile balance (HLB) of, for example, from about 12 to about 17. The quantity of surfactant in such formulations will typically range from, for example, about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Injectable formulations are in accordance with the present invention. The requirements for effective pharmaceutical carriers for injectable compositions are well-known to those of ordinary skill in the art (see, e.g., Pharmaceutics and Pharmacy Practice, J.B. Lippincott Company, Philadelphia, Pa., Banker and Chalmers, eds., pages 238-250 (1982), and ASHP Handbook on Injectable Drugs, Trissel, 14th ed., (2007)).

In accordance with the methods of the present invention, the attending physician will decide the dosage of the alphaRPT and imaging surrogates with which to treat each individual patient, taking into consideration a variety of factors, such as age, body weight, general health, diet, sex, amount to be administered, route of administration, and the severity of the condition being treated.

In an embodiment, the alphaRPT and imaging surrogates are mixed with excipients which are pharmaceutically acceptable and compatible with the active ingredient.

In accordance with one or more embodiments, the imaging surrogate compositions of described herein can be used to determine the appropriate dosage level of an alphaRPT of interest in a subject comprising: a) administering the imaging surrogate to the subject; b) measuring the pharmacokinetic profile of the imaging surrogate in the subject using imaging; and c) if necessary, performing pharmacokinetic modeling to account for differences between the imaging surrogate and the alphaRPT; and d) using the information from b) and/or c) to determine the appropriate dosage level of the alphaRPT of interest for the subject.

In accordance with another embodiment the present invention provides a method for determining an appropriate dosage level of an alphaRPT of interest in a subject comprising: a) administering an imaging surrogate composition to the subject, wherein the imaging surrogate comprises ¹¹¹In or ⁶⁸Ga or ⁸⁹Zr conjugated to a targeting moiety, and wherein the imaging surrogate composition has a pharmacokinetic profile which is significantly similar to an alphaRPT conjugated to the same targeting moiety labeled with an alpha-emitting isotope selected from the group consisting of ²¹³Bi, ²²⁵Ac and ²¹²Pb; b) measuring the pharmacokinetic profile of the imaging surrogate in the subject using imaging; c) if necessary, performing pharmacokinetic modeling to account for differences between the imaging surrogate and the alphaRPT; and d) determining the appropriate dosage level of the alphaRPT of interest for the subject.

In accordance with one or more embodiments of the present invention, imaging measurements are compared with the tissue counting data. Pharmacokinetic studies are performed for tumor-bearing mice treated with and without an approach that blocks the targeted receptors; PK following aPC with excess unlabeled PC will also be obtained to demonstrate targeting specificity. Treated mice receive a mixture of both aPC (100 nCi) and iPC (5 μCi) via IP injection. At 5, 15, 30 minutes, 1, 2, 4, 6, 24, 72 and 120 hours 5 mice are sacrificed at each time point and blood, kidneys, lungs, heart, liver, brain, spleen, stomach, muscle, tumor and the remaining carcass are assayed for radioactivity. Energy windows are set on the gamma counter to simultaneously count both ¹¹¹In and ²²⁵Ac; counts for ²¹³Bi, the 45.6-min-half-life descendant of ²²⁵Ac, are obtained by counting tissue samples immediately after collection (before excess ²¹³Bi in tissue has decayed) and after 24 hours (while ²¹³Bi is in radioactive equilibrium with ²²⁵Ac).

Imaging is performed on 3 mice (imaged simultaneously) at 5, 30 minutes and 1, 2, 3, 24, 72, 120 hours post-injection w/iPC; imaging studies are also performed with and without IV blocking. The resulting time-activity data is used to calculate the absorbed dose to tumor and normal organs from aPC. Absorbed doses are scaled by the activity level required for organ toxicity or anti-tumor efficacy.

In accordance with another embodiment, the present invention provides a method for determining an appropriate dosage level of an alphaRPT of interest in a subject comprising: a) administering an imaging surrogate composition to the subject, wherein the imaging surrogate comprises ¹³¹I conjugated to a targeting moiety, and wherein the imaging surrogate composition has a pharmacokinetic profile which is significantly similar to an alphaRPT conjugated to the same targeting moiety labeled with ²¹¹At; b) measuring the pharmacokinetic profile of the imaging surrogate in the subject using imaging; c) if necessary, performing pharmacokinetic modeling to account for differences between the imaging surrogate and the alphaRPT; and d) determining the appropriate dosage level of the alphaRPT of interest for the subject.

In accordance with a further embodiment, the present invention provides a method for determining an appropriate dosage level of an alphaRPT of interest in a subject comprising: a) administering an imaging surrogate composition to the subject, wherein the imaging surrogate comprises ⁸⁹Zr conjugated to a targeting moiety, and wherein the imaging surrogate composition has a pharmacokinetic profile which is significantly similar to an alphaRPT conjugated to the same targeting moiety labeled with ²²⁵Ac; b) measuring the pharmacokinetic profile of the imaging surrogate in the subject using imaging; c) if necessary, performing pharmacokinetic modeling to account for differences between the imaging surrogate and the alphaRPT; and d) determining the appropriate dosage level of the alphaRPT of interest for the subject.

It will be understood by those of ordinary skill in the art that the methods of the present invention are not limited to the alpha-emitting isotopes discussed herein. Other alpha-emitting isotopes, such as, ²²³Ra and ²²⁹Th, for example, and others which may be developed in the future, can be used in accordance with the present invention.

### Examples

[¹¹¹-In]-DOTA-substance P radiolabeling. All solutions and water are prepared in plastic (polystyrene) bottles. About 1.0 L deionized water was prepared with Millipore Simplicity 185 system. 5 grams of Chelex-100 resin (Bio-rad cat. #142-2842) is added to deionized water, mixed well and left at room temperature for 10 minutes with occasional shaking. The Chelex-100 water was then filtered with Millipore vacuum filtering unit (polyethersulfone membrane, Millipore cat. # SC56PU1ORE), and stored at 4° C. All solutions are prepared with chelexed water.

About 6.56 g sodium acetate (J.T. Baker cat.#3479-01). Add 35 ml chelexed water and mix to dissolve. Using a pH meter (Corning pH meter 430) to measure the pH of the buffer solution. Add acetic acid (Fisher scientific, cat. # UN2789) to adjust the pH of the buffer to pH=6.5, and add chelexed water to a final volume of 40 ml. About 0.5 g of Chelex-100 resin is added to the 2 M sodium acetate buffer solution, mixed and allowed to stand 10 minutes, followed by filtration with a Poly-Prep chromatography column (Bio-rad cat. #731-1550). The sodium acetate stock solution (2 M, pH=6.5) is stored at room temperature.

DOTA-substance P powder was added to chelexed water to a final concentration of 5 mg/ml. About 1 ml stock sodium acetate buffer (2 M, pH=6.5) was mixed with 19 ml chelexed water. (*Concentration of reaction buffer is adjusted based on the amount of ¹¹¹InCl₂ used in the reaction.)

Radioprotectant solution (ascorbic acid buffer) was prepared by adding about 1.5 g ascorbic acid (Sigma cat. #255564) to 10 ml chelexed water to a final concentration of 150 mg/ml and mixed. Prepare fresh ascorbic acid buffer before radiolabeling.

Radiolabeling DOTA-substance P with ¹¹¹In was performed as follows. A heating block was preheated to 95° C. 40 μl 0.1 M sodium acetate buffer was added to the reaction tube (1.7 ml microcentrifuge tubes, Denville, cat. #C-2170), followed by addition of ¹¹¹InCl₂ to the reaction buffer. The radioactivity is measured with a dose calibrator (Capintec CRC-15C). About 2 μl (10 μg) of stock DOTA substance P solution (5 mg/ml) is added to the reaction buffer and mixed. The solution is incubated in the heating block at 95° C. for 5 minutes, and then removed from the heating block and allowed to cool at room temperature for 5 minutes.

Instant thin layer chromatography of ¹¹¹In radiolabeled DOTA-substance P. 10 mM EDTA solution is placed in a glass beaker (also in a separate glass beaker with (9% NaCl/10 mM NaOH) until it is about 0.5 cm deep. About 1 μl radiolabeled peptide is placed on the chromatographic strip (silica gel impregnated pap, Agilent Technology) about 1 cm from the bottom. The strip is placed in the glass beaker and the solvent is allowed to run up the strip. When solvent is about 5 mm from the top, the strip is removed and cut the strips into two equal parts (upper and lower half). The two halves are counted separately in a gamma counter (LKB Wallac 1282 CommpuGamma CS). For 10 mM EDTA migrated solution, % of free radionuclide=(counts on upper half)/(counts on lower half+counts on upper half)*100%. For 9% NaCl/10 mM NaOH migrated solution, % of un-chelated radionuclide=(counts on lower half)/(counts on lower half+counts on upper half)*100%. The peptide labeling efficiency was calculated as =100%−% peptide unbound radioactivity-% insoluble hydroxide.

HPLC quality control of the radiolabeled peptide. The quality of ¹¹¹In/²²⁵Ac-radiolabeled substance P was verified using RP-HPLC Beckman coulter Gold 126 connected to a Jupiter C18 column (Phenomenex). The radiolabeled peptide was acquired using the following separation method. Solvent A: 0.1% trifluoric acid (vol/vol) in water; solvent B: 0.1% trifluoric acid (vol/vol) in acetonitrile. The gradient is set up as follows: 0-20 minutes, 5-60% solvent B; 20-25 minutes, hold at 60% solvent B; 25-30 minutes, 60-90% solvent B; 30-35 minutes, 90-5% solvent B. The Substance P peak is eluted as a single peak around 15 minutes. This completes the manufacture and control of ¹¹¹In-DOTA-substance P.

Radiolabeling DOTA-substance P with ²²⁵Ac. Preheat heating block to 95° C. About 40 μl 0.1M sodium acetate buffer is added to the reaction tube (1.7 ml microcentrifuge tubes, Denville, cat. #C-2170) followed by the addition of about 1-5 μl 150 mg/ml ascorbic acid as radioprotector. The ²²⁵Ac is then added to the reaction buffer, and radioactivity is measured with a dose calibrator (Capintec CRC-15C, setting 775, times five). About 2 μl (10 μg) stock DOTA substance P solution (5 mg/ml) is added to the reaction buffer and mixed, followed by incubation in the heating block at 95° C. for 5 minutes. The reaction tube is removed from the heating block and allowed to cool at room temperature for 5 minutes.

Instant thin layer chromatography of DOTA-substance P with ²²⁵Ac and HPLC quality check of radiolabeled peptide is performed as previously described combining HPLC and TLC analysis.

The determination of the pharmacokinetics of ¹¹¹In-labeled peptide conjugate (iPC) and ²²⁵Ac-labeled peptide conjugate (aPC). aPC and iPC are characterized by a combination of direct tissue sampling (aPC and iPC) and small animal (SPECT/CT) imaging (iPC only). The former provides quantitative evaluation of kinetics as the average measurement from multiple animals at each time-point and allows direct comparison of aPC with iPC while the latter provides longitudinal measurements in each of the imaged animals that will be used to identify sites of iPC accumulation not anticipated by tissue sampling.

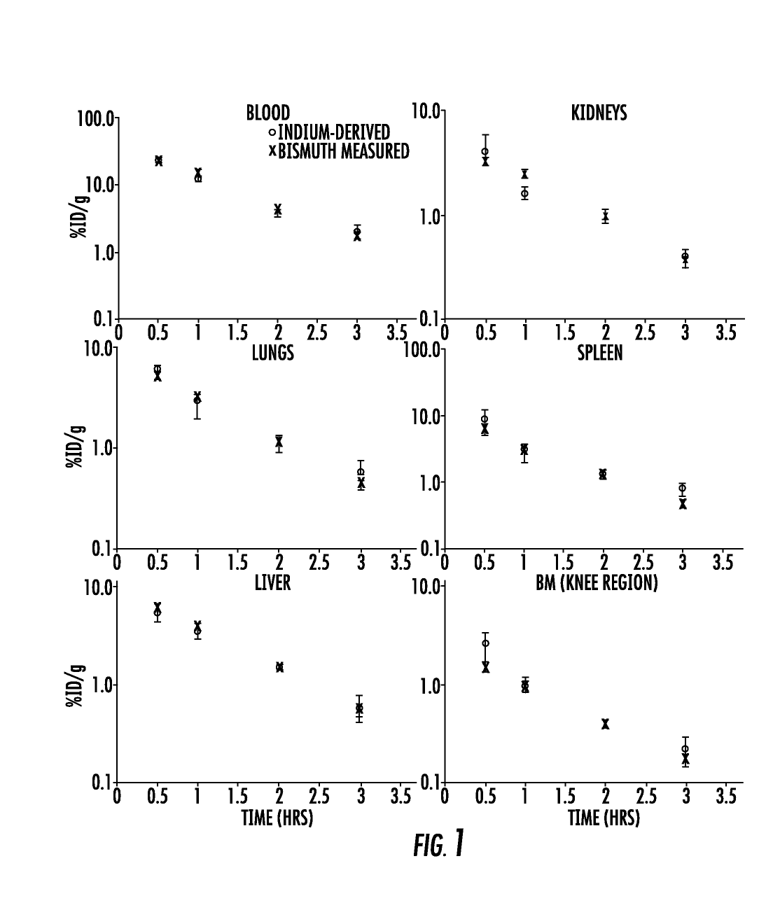

¹¹¹In- vs. ²¹³Bi-labeled and ²²⁵Ac-labeled antibody. To determine whether the claimed methods of the present invention would show that an ¹¹¹In-labeled anti-HER2/neu Ab is an adequate imaging surrogate for ²¹³Bi-Ab, the following experiment was performed. Tumor free mice were injected with either the ¹¹¹In-labeled anti-HER2/neu Ab or the ²¹³Bi-Ab. The blood and organs of the mice were then harvested at various time points and time-activity curves were plotted (FIG. 1). As shown by the overlap in points, the biodistribution of ¹¹¹In-labeled 7.16.4 antibody does indeed, closely match that of the ²¹³Bi-labeled antibody and the time-integral of the curves are approximately the same.

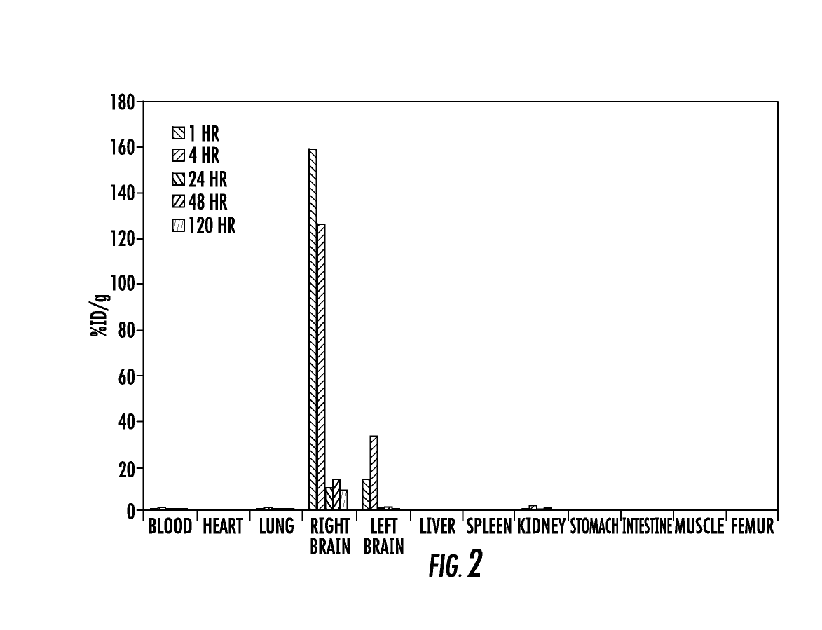

FIG. 2 shows similar results for ²²⁵Ac-labeled peptide with ¹¹¹In again as the surrogate imaging agent.

Clinical Application. The pharmacokinetics of an analogous ¹¹¹In-labeled peptide conjugate (iPC) will be administered to subjects and compare the kinetics and biodistribution with that of ²²⁵Ac-labeled peptide conjugate (aPC). The data collected will enable modeling so that iPC PK may be used to derive aPC PK. The PK obtained from iPC measurements can then be used to calculate aPC dosimetry (aPC cannot be directly imaged because the activity administered for therapy will be below the sensitivity of imaging equipment). The PK obtained using iPC will be “credentialed” as an appropriate surrogate for aPC by demonstrating that iPC predicts aPC tumor and normal organ dosimetry under each of the treatment conditions investigated.

Using canine OS models to evaluate alphaRPT efficacy and toxicity in humans. In the sarcoma field, canine OS is widely regarded to be the best model of human OS. As in humans, OS in dogs occurs primarily in the long bones (˜75% of cases), and less commonly in the axial skeleton. Amputation alone provides good local control but within one year 90% of dogs not treated with some form of systemic therapy will develop metastases, usually to the lungs. In humans, 80% will develop metastases, also usually to the lungs, 2-years after amputation if no systemic therapy is given. Canine and human OS histology are almost identical; both are likely to be high-grade with rapidly metastasizing tumors. Furthermore, canine OS occurs predominantly in large breed (>35-40 kg) dogs, enabling imaging and PK studies that are essential to a treatment planning implementation of alphaRPT. We have incorporated into the canine trial design the treatment planning approach that will be essential for αRPT of human OS.

In accordance with one or more embodiments, alphaRPT will be performed using ²²⁵Ac-labeled trastuzumab. This approach obviates the limitations of the prior art therapies. The high-energy α-particles emitted by ²²⁵Ac cause largely irreparable double-strand DNA damage; such damage is impervious to chemo- and radio-resistance. Anti-HER2/neu Ab mediated delivery of ²²⁵Ac will target all OS cells, including soft-tissue lesions.

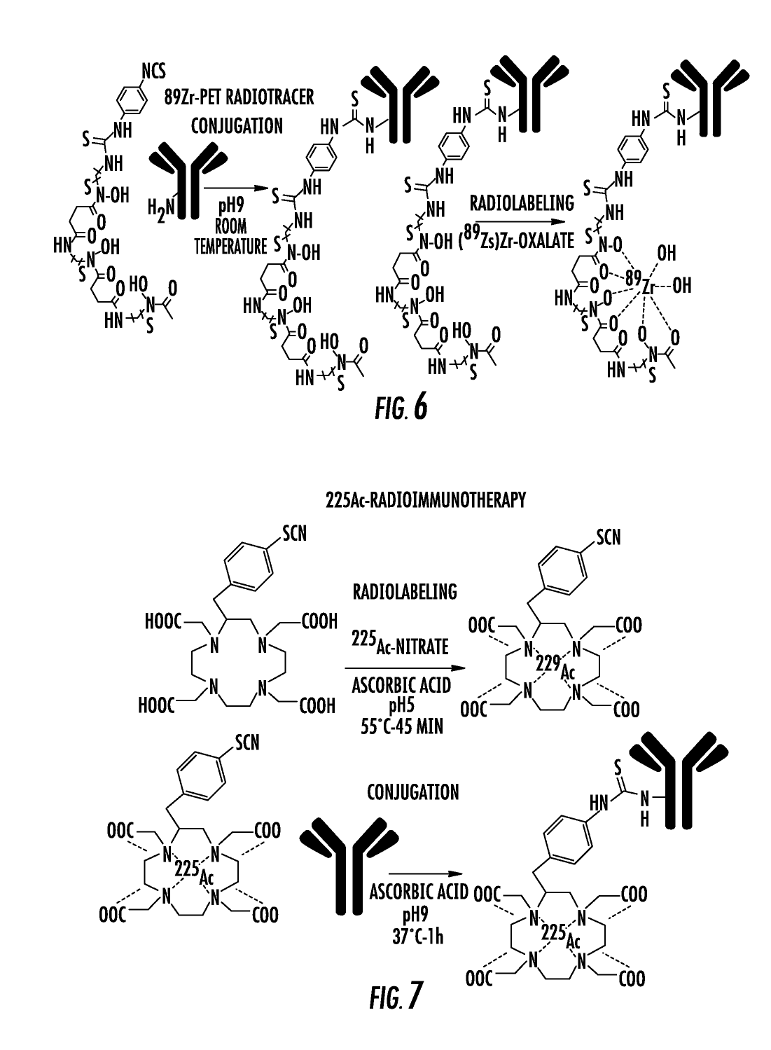

Radiochemistry: [⁸⁹Zr]Zr-DFO-trastuzumab is prepared following the protocol reported by Vosjan et al. (Nature protocols. April 2010; 5(4):739-743) (FIG. 6). DFO-Ab conjugate is purified utilizing centrifugation filter unit (30 KDa molecular weight cut-off) in order to separate the remaining unconjugated DFO chelate from the modified Ab. DFO-immunoconjugate quality control will be evaluated utilizing HPLC. Following the chelator attachment, ⁸⁹Zr-radiolabeling will be undertaken starting from a source of [⁸⁹Zr]Zr-oxalate 1 M. The radiotracer is then purified using centrifugation filtration units. The final radiolabeled product will be characterized using Instant Thin Layer Chromatography (ITLC) migrated with 10 mM EDTA. The migration ratio radioprobe to solvent is expected to be Rf=0 for a radiolabeling conducted to completion.

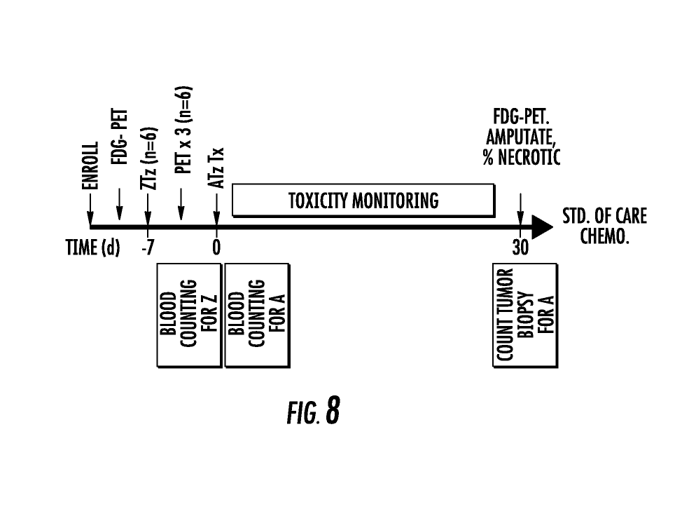

²²⁵Ac-DOTA-Trastuzumab is prepared as previously described by Mc Devitt et al. (Applied radiation and isotopes: including data, instrumentation and methods for use in agriculture, industry and medicine. December 2002; 57(6):841-847) (FIG. 7). DOTA-NCS conjugate is radiolabeled with a hydrated solution of ²²⁵Ac-nitrate at 55° C. and 40 minutes. The radiocomplex purity is verified using cationic exchange chromatography and ITLC. The resultant complex ²²⁵Ac-DOTA-NCS is migrated on TLC strips using either EDTA (10 mM) or NaOH (10 mM), NaCl (150 mM) to check if ²²⁵Ac chelation was completed. ²²⁵Ac-DOTA radiocomplex is then conjugated to the antibody. The preparation is purified using a desalting size exclusion chromatography column. The resultant product is tested using ITLC by strips migration dissolved in EDTA (10 mM) or NaOH (10 mM), NaCl (150 mM). ²²⁵Ac-labeled trastuzumab is considered pure when both strips present a migration ratio radioactivity to solvent Rf=0. All preparations for sterile injections are solvated in sterile physiological saline concentration and at physiological pH. Limulus amebocyte lysate testing will be used to determine pyrogen content, and microbiologic culture in fluid thioglycollate of soybean casein digest medium to verify sterility.

In accordance with the inventive methods, the inventors will identify the MTD while also collecting data that will allow calculation of DLO and tumor dosimetry. There is already considerable experience with ²²⁵Ac-labeled antibody, but the experience does not provide accurate, imaging-based estimates of organ absorbed dose. The MTD estimates described in the literature do not apply to canines with OS. This is because the tumor burden in dogs is generally lower than in leukemia patients, it is less rapidly accessible and therefore not likely to be as substantial a sink that removes alphaRPT from the circulation. Accordingly, the MTD in humans is likely an overestimate of the MTD in canines. The MTD in tumor-free monkeys is an underestimate for the opposite reason—there is no disease to take up alphaRPT in circulation. No study used imaging as in accordance with the inventive methods because the activity of ²²⁵Ac was too low to image. As a result the MTD that is expressed in terms of AA cannot be related to the AD in marrow (acute DLO) and kidney (delayed DLO). Using the inventive methods the MTD is identified in tumor-bearing dogs and the imaging and PK data is collected which will relate DLO AD to measured hematologic and renal toxicity.

A phase I/II type protocol, similar to that used for human studies when the number of patients is limited will be used and is depicted in FIG. 8. Toxicity will be investigated at 2, 3 and 4 μCi/kg. Blood counts, blood urea nitrogen (BUN), creatinine and urine specific gravity (USG) will be monitored to evaluate toxicity. A total of six dogs will be imaged; one at each dose level will be IV injected with ZTz one week prior to receiving ATz. PET/CT images will be collected at 24, 72 and 96 hours after ZTz administration. Blood samples will be collected at 1, 4, 24, 72 and 96 hours after administration of ZTz. The same blood collection and counting protocol will also be observed following ATz administration for dogs that will not be imaged. The ZTz and ATz data will allow us to compare PK of ATz and its surrogate ZTz in blood. ZTz will track antibody-conjugated ²²⁵Ac but not the ²¹³Bi daughters. Circulating levels of free ²¹³Bi will be measured by counting immediately after sample collection and 5 to 24 hours later when ²²⁵Ac is in equilibrium with its daughters. By comparing early to late counts, uptake or reduced accumulation of daughters in different tissues can be derived. After 30 days, all dogs will undergo amputation and a tumor biopsy will be obtained for counting, the tumor will also be used to evaluate efficacy and compare ATz vs ZTz uptake. The dogs will then proceed to standard of care chemotherapy.

The longitudinal PET/CT images will be transferred to Hopkins and used in a 3D-RD calculation that uses canine anatomy, as defined by the CT, to perform a Monte Carlo calculation of absorbed dose (Semin Nucl Med. September 2008; 38(5):321-334). The 3D-RD methodology does not use S values (MIRD Pamphlet No. 11. New York: Society of Nuclear Medicine; 1975). S values assume an idealized human anatomy and are applicable to risk-related dosimetry calculations for diagnostic imaging (J Nucl. Med. March 2009; 50(3):477-484). The 3D-RD dose estimates will be individualized to the specific anatomy and pharmacokinetics of each dog imaged.

The AA-based MTD will be identified and will subsequently use the PK/imaging data collected to relate dose-limiting organ AD to measured toxicity. The dose associated with a 10% DLT probability will be determined, where a DLT is defined as Grade 3 or above acute hematologic toxicity as well as renal toxicity based on Common Terminology Criteria for Adverse Events (CTCAE). The final estimate of the MTD is the AA in μCi/kg with a DLT rate closest to the target rate of 10%. Secondary objectives are to utilize PET imaging, collect PK data to relate dose-limiting organ (DLO) absorbed dose (AD) to measured toxicity.

¹¹¹In-Tz was previously used as an imaging and biodistribution surrogate for ²¹³Bi-Tz and has shown identical distribution. In accordance with the inventive methods, ⁸⁹Zr-Tz will be used because it can be imaged by PET and will yield higher resolution and more quantitative data than ¹¹¹In-SPECT. Since ZTz will be used to track the distribution of ²²⁵Ac-Tz and not the daughters, the stability of both constructs, in vivo, is key to having one act as a surrogate of the other. The radiochemical stability, in vivo, of ZTz is well established and this agent has been used in humans. Numerous ²²⁵Ac-Ab conjugates have been developed, including with trastuzumab, and their stability is well established. We will confirm that ZTz is a PK/imaging surrogate of ATz by comparing blood PK. We will also compare imaging-based ZTz predictions of tumor targeting with counting of tumor samples for ATz. If the kinetics in blood differ, these differences will be modeled and use model-based PK to adjust ZTz tissue uptake and clearance so that it reflects ATz uptake and clearance. For example, if ATz clears faster than ZTz in circulation we can account for this in a PK model by increasing the clearance rate of ATz until it matches measured blood kinetics. We expect that toxicity will be minimal at the first, 2 μCi/kg, dose level. At 3 μCi/kg we expect a 10% likelihood of encountering renal toxicity and 30% likelihood at 4 μCi/kg. At the end of this procedure we will have identified the MTD. By imaging and PK analysis, we will also have the first reliable calculation of marrow and kidney absorbed dose at the MTD.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An imaging surrogate composition comprising:
a) at least one detectable moiety conjugated to a targeting moiety, wherein the imaging surrogate composition has a pharmacokinetic profile which is significantly similar to an alpha-emitting radionuclide conjugated to the same targeting moiety (alphaRPT);
b) wherein the pharmacokinetic profile of the imaging surrogate is determined by comparing the pharmacokinetic profile of the imaging surrogate to the pharmacokinetic profile of the alphaRPT in one or more preclinical models; and
c) wherein the imaging surrogate composition derived alphaRPT dosimetry is within 1-20% of directly calculated alphaRPT dosimetry to a dose-limiting organ in said one or more preclinical models.

2.-3. (canceled)

4. The imaging surrogate composition of claim 1, wherein the detectable moiety of the imaging surrogate composition is selected from the group consisting of ¹⁸F, ¹¹C, ¹³N and ¹⁵O, ⁷⁵Br, ⁷⁶Br, ¹²⁴I, ²⁰¹Tl, ^{99m}Tc, ¹²³I, ¹¹¹In, ⁶⁸Ga, ⁶⁴Cu, ⁸⁹Zr and ¹³¹I.

5. The imaging surrogate composition of claim 1, wherein the imaging surrogate composition comprises ¹¹¹In and has a pharmacokinetic profile which is significantly similar to an alphaRPT comprising ²¹³Bi, or ²²⁵Ac, or ²¹²Pb.

6. The imaging surrogate composition of claim 1, wherein the imaging surrogate composition is elected from the group consisting of ¹³¹I, ¹²³I, or ¹²⁴I and has a pharmacokinetic profile which is significantly similar to an alphaRPT comprising ²¹¹At.

7. The imaging surrogate composition of claim 6, wherein the imaging surrogate composition comprises ¹³¹I and has a pharmacokinetic profile which is significantly similar to an alphaRPT comprising ²¹¹At.

8. The imaging surrogate composition of claim 1, wherein the imaging surrogate composition comprises ⁸⁹Zr and has a pharmacokinetic profile which is significantly similar to an alphaRPT comprising ²²⁵Ac.

9.-12. (canceled)

13. A method for determining an appropriate dosage level of an alphaRPT of interest in a subject comprising:
a) administering an imaging surrogate composition to the subject, wherein the imaging surrogate comprises at least one detectable moiety conjugated to a targeting moiety, and wherein the imaging surrogate composition has a pharmacokinetic profile which is significantly similar to an alphaRPT comprising the same targeting moiety labeled with alpha-emitting isotope;
b) measuring the pharmacokinetic profile of the imaging surrogate composition in the subject using radionuclide imaging;
c) if necessary, performing pharmacokinetic modeling to account for differences between the imaging surrogate composition and the alphaRPT; and
d) determining the appropriate dosage level of the alphaRPT of interest for the subject.

14. the method of claim 13,
wherein the imaging surrogate comprises ¹¹¹In or ⁶⁸Ga or ⁸⁹Zr conjugated to a targeting moiety, and wherein the imaging surrogate composition has a pharmacokinetic profile which is significantly similar to an alphaRPT conjugated to the same targeting moiety labeled with an alpha-emitting isotope selected from the group consisting of ²¹³Bi, ²²⁵Ac and ²¹²Pb.

15. the method of claim 13,
a wherein the imaging surrogate comprises ¹³¹I conjugated to a targeting moiety, and wherein the imaging surrogate composition has a pharmacokinetic profile which is significantly similar to an alphaRPT conjugated to the same targeting moiety labeled with ²¹¹At.

16. A method of treatment of a disease in a subject comprising:
a) administering an imaging surrogate composition to the subject, wherein the imaging surrogate comprises at least one detectable moiety conjugated to a targeting moiety, and wherein the imaging surrogate composition has a pharmacokinetic profile which is significantly similar to an alphaRPT comprising the same targeting moiety labeled with alpha-emitting isotope;
b) measuring the pharmacokinetic profile of the imaging surrogate in the subject using imaging;
c) if necessary, performing pharmacokinetic modeling to account for differences between the imaging surrogate and the alphaRPT;
d) using the information from b) and/or c) to determine the appropriate dosage level of the alphaRPT of interest for the subject; and
e) administering to the subject the alphaRPT of interest at the dosage level determined in d) to treat the disease in the subject.

17. The method of claim 16, wherein the disease is selected from the group consisting of cancer, autoimmune disease, trigeminal neuralgia, acoustic neuromas, severe thyroid eye disease, pterygium, pigmented villonodular synovitis, atherosclerotic plaques, prevention of keloid scar growth, vascular restenosis, and heterotopic ossification.

18. The method of claim 16, wherein the disease is cancer and the imaging surrogate is a targeting moiety conjugated with ¹³¹I or ¹²³I or ¹²⁴I, and wherein the alphaRPT comprises the same targeting moiety conjugated with an alpha-emitting isotope selected from the group consisting of ²¹³Bi, ²²⁵Ac and ²¹²Pb.

19. The method of claim 16, wherein the disease is cancer and the imaging surrogate is a targeting moiety conjugated with ¹³¹I or ¹²³I or ¹²⁴I, and wherein the alphaRPT comprises the same targeting moiety conjugated with ²¹¹At.

20. The method of claim 16, wherein the disease is OS and the imaging surrogate is [⁸⁹Zr]Zr-DFO-trastuzumab and the alphaRPT is ²²⁵Ac-DOTA-trastuzumab.

21. The method of claim 16, wherein the pharmacokinetic profile of the imaging surrogate in the subject is measured using planar or SPECT or PET imaging.
